(19) United States
(12) Patent Application Publication
(10) Pub. No.: US 2014/0323851 A1
(43) Pub. Date: Oct. 30, 2014

(54) SUBJECT PLACEMENT AND HEAD POSITIONING DEVICE

(71) Applicant: XLR IMAGING INC., London (CA)

(72) Inventors: Enzo Antonio Barberi, London (CA); Joseph Robert Kerr, London (CA)

(21) Appl. No.: 14/360,511

(22) PCT Filed: Nov. 23, 2012
(86) PCT No.: PCT/CA2012/001083

§ 371 Date: May 23, 2014

Related U.S. Application Data
(60) Provisional application No. 61/563,709, filed on Nov. 25, 2011.

(51) Int. Cl. A61B 5/055; A61G 13/12

(52) U.S. Cl. CPC A61B 5/0555; A61G 13/121
USPC 600/415; 5/622

(57) ABSTRACT

The present invention relates to a subject placement device for use in a diagnostic and/or therapeutical treatment setting. The subject placement device of the present invention includes: (a) a board for receiving a body of the subject, and (b) a head cradle extending from said board, said head cradle being enabled to receive a range of head diameters.

### CROSS-REFERENCE TO RELATED APPLICATION

This application claims priority to U.S. provisional application No. 61/563,709 filed on Nov. 25, 2011, which is incorporated herein in its entirety by reference.

### FIELD OF THE INVENTION

The present invention relates generally to patient placement in a medical diagnostic and/or treatment setting and, more specifically, to a patient placement device having a head cradle that reduces head motion and allows for accurate, repeatable head position centering when used in diagnostic imaging or radiation oncology applications.

### BACKGROUND OF THE INVENTION

Diagnostic imaging, which may include magnetic resonance imaging (MRI), computed tomography (CT), positron emission tomography (PET), or X-rays, and radiation oncology require accurate and consistent placement of anatomical regions of interest for patients of various ages and sizes relative to a desired volume, which is defined by a three dimensional imaging field of view or radiation target. There are also applications in diagnostic imaging and oncology that require the preparation of a subject outside the exam room, such as in a preparatory room or on a gurney.

In diagnostic imaging and/or oncology treatments, patient/subjects are placed into a landmark location at the center of an imaging system or radiation dosing site through the use of a sliding patient table. The head is then typically positioned in a head cradle or equivalent to assist in head land marking and position registration.

Currently, manufacturers of diagnostic imaging and radiation oncology systems design their systems to accommodate a maximum patient size, and then rely on highly trained diagnostic imaging and/or radiation oncology technicians to accurately place patients in their systems, often on the patient table, with basic and generic positioning pads and ad-hoc tools such as foam wedges and small fabric rolls, so as to accommodate the various ages and head sizes for acceptable patient placement. This procedure is often a time consuming and iterative process, particularly for applications where: wide variations in subject size are common, the patient is non-compliant, sedated or anesthetized, and/or the patient needs to be prepared outside of the exam room.

One diagnostic imaging modality of particular interest is MRI, which has several advantages over other diagnostic imaging modalities, including: superior imaging detail; increased sensitivity and specificity of diagnosing neurological disease; and the ability to assess brain development and function without the use of ionizing radiation. However, this modality also presents unique challenges that are specifically addressed by the current invention,

Magnetic resonance imaging requires the presence of an electromagnetic (EM) device to transmit and receive non-ionizing radiation from the subject or patient. These devices are commonly called RF coils; “RF” since the frequency of the non-ionizing radiation is in the radio frequency (RF) band, and “coil” because of the original, simple inductor coil designs from early historical Nuclear Magnetic Spectroscopy studies of chemical samples in vials from the 1940's.

The large distance from the MRI volume of interest (VOI) to the “stock” RF body coil (in the magnet bore) decreases their sensitivity and as a result, creates poor images in comparison to coils that are in close proximity to the VOI. Therefore, it is quite common to have RF coils attached or located closer to the subject for faster, higher resolution imaging studies. The positioning of coils on the right location of the subject is of great importance in the setup before MRI scans. In particular, conformal, multi-channel RF “head coils”, where the mechanical structure is designed to envelope the patient's head as closely as practical, are known to provide the fastest and highest resolution imaging of the head region. Patient comfort and ease of patient placement and removal from conformal RF coils is an issue for the preparatory setup required for human subject MRI scans.

Furthermore, due to the nature of how MR images are obtained, the reduction of motion of subjects is critical to ensure high quality MR images, and this problem can be a particular issue with conformal coils that do not fit the patient tightly.

Because of its lack of ionizing radiation, MRI has become the preferred diagnostic imaging modality of choice for pediatrics. Unfortunately, pediatric use can exacerbate the preparatory and motion problems of MR imaging (and indeed other modalities) in general and specifically when done using conformal coils. MRI scans involve a significant use of time and resources to obtain clinically relevant diagnostic images, typically 30 to 60 minutes, and require patients/subjects to lie still for the entire exam, which is difficult for young children to comply with, particularly for infants and children under the age of 6 years. As a result, current practice at pediatric hospitals and imaging centers involves the administration of sedatives or anesthetics to children, which greatly increases the risk to patients, the total exam time required, and the cost of the MRI exam. A recent study shows that the use of sedation and anesthetics to add 3 to 10 times to the cost for the MRI exam, respectively (Effect of Anesthesia and Sedation on Pediatric MR Imaging Patient Flow, Radiology, Vol. 256, No. 1, July 2010). However, one clinical study shows that if the total MRI exam for pediatric subjects is kept below 30 minutes, the use of sedation and anesthetics drops from approximately 75% to below 10% for children aged 3-5 years (Growth Rates in Pediatric Diagnostic Imaging and Sedation, Anesthesia & Analgesia Vol. 108, No. 5, May 2009). It is therefore highly desirable to implement techniques and technologies that reduce the overall exam time to less than 30 minutes and reduce child motion, while still generating clinically acceptable images.

Recent advances in RF coil technology employ the aforementioned conformal designs to place coil elements as close as possible to the pediatric patient for faster, higher resolution imaging studies for total exams in less than 30 minutes. Unfortunately, the conformal nature of these coils presents challenges in placing and removing a subject's head, due to the limited space between the coil and head. The current practice to address this issue is to use improvised solutions and oversized coils, which take additional time and reduce the potential savings that would accrue from using appropriately sized RF head coils for the child.

In pediatric imaging, and in particular MR imaging, a child that is sedated or anesthetized (non-compliant) adds additional difficulties, including: in head positioning and placement m conformal coils; in transferring the child from a preparatory room/gurney to the imaging system; and in removing the subject from the conformal coils.

The following inventions address some of the functions involved with positioning and placement of patients/subjects, particularly children, for diagnostic imaging and/or oncological treatment applications.

Patient Transfer and Positioning

U.S. Pat. No. 7,467,004 to Calderon discloses a surgical compatible patient table capable of sliding a patient and RF coil system into an MRI system, with the RF coil acting as the head cradle.

U.S. Pat. No. 8,276,225 to Kogan discloses a similar sliding table patient positioning system with an integrated, recessed head cradle capable of sliding a patient into a nuclear medicine imaging system, such as PET, SPECT or Gamma camera based systems.

U.S. Pat. No. 7,752,687 to Denosky discloses a mobile patient transfer device for transporting patients to Diagnostic Imaging or Medical Treatment systems with a sliding patient table, and transferring patients onto the sliding patient table.

Patient Body and Head Positioning Systems

U.S. Pat. No. 8,093,569 to Miller discloses a modular patient support system for accurately delivering radiation to a targeted site within a cancer patient utilizing a patient pod and a rigid moldable foam head cradle.

U.S. Pat. No. 7,013,171 to Rohdes discloses a medical imaging method utilizing foam pads for supporting the head and neck of a supine patient during medical imaging.

U.S. Pat. No. 7,213,596 to Saied discloses a patient positioning device comprising a base and various inflatable chambers to adjust head position and tilt to facilitate insertion and proper placement of a laryngoscope blade into the mouth of a patient.

U.S. Pat. No. 7,450,985 to Meloy discloses a head restraint kit particularly suited for use with a head coil for magnetic resonance imaging. The head restraint kit contains a head rest pillow, a neck cushion, and one or more wedges for providing additional support, and a cloth strap placed over the forehead of a patient for protecting and securing the head against movement.

U.S. Pat. No. 5,388,580 to Sullivan discloses a non-metallic MRI compatible adjustable head holder and fixation device for preventing head rotation or translation along any of three axes.

U.S. Pat. No. 5,263,494 to Margelos discloses a vertical head positioner for cephalometric x-ray.

U.S. Pat. No. 6,656,143 to Browd discloses a vacuum fixation device to provide semi-rigid head fixation during medical imaging and medical procedures/operations of the face and head.

U.S. Pat. No. 7,984,715 to Moyers discloses a registration and immobilization device for the head and neck region of patients undergoing radiation therapy, comprised of a support shell shaped to conform to the shape of the head and neck, combined with a conformal face mask that serves to effectively clamp the face and head down to the support shell.

U.S. Pat. No. 5,515,867 to Lamb discloses a patient head support for a shoulder surgery positioner having a head rest/cradle and forehead and chin straps for immobilization.

U.S. Pat. No. 8,245,335 to Shvartsberg discloses a structural component such as a head clamp for use in imaging of a part of a patient using Magnetic Resonance Imaging and X-ray imaging.

U.S. Pat. No. 6,143,003 to Cosman discloses a repositioner for head, neck and body, permitting repeat localization and immobilization of the bead, neck and/or torso of a patient's body, comprising immobilizing structures used to secure the head and neck and torso in a relative orientation.

U.S. Pat. No. 8,146,599 to Wilson discloses a patient positioning system on a treatment couch top for Stereotactic Body Radiation Therapy, comprising positioning/fixation components including straps and foam supports.

Patient and Equipment Positioning Systems

U.S. Pat. No. 5,085,219 to Ortendahl discloses an adjustable holder for MRI RF surface coils and a foam head cradle with restraining straps.

U.S. Pat. No. 8,131,341 to Heumann discloses a method and device for positioning a patient support apparatus that has a mounting for accommodation of RI coil devices at predetermined locations.

Pediatric Designs for Diagnostic and/or Oncological Treatment Applications:

U.S. Pat. No. D446,675 to Straub discloses an ornamental design for infant head positioner, comprised of wedges and a base.

Disadvantages of the positioners and head cradles of the prior art disclosures include; (1) they are too cumbersome and invasive; (2) involve patient compliance which is not possible with children; (3) require relatively long time to set up and to use (a particular disadvantage for child patients); (4) cannot handle the variation in head sizes present in the general population and children; and (5) have mechanisms that are too visible to children (appearance has an impact on function since it affects acceptance from young children, which affects sedation use.

### SUMMARY OF THE INVENTION

The present invention addresses the positioning and placement functions required for diagnostic imaging and/or therapeutic treatment applications in a unique manner that is different from the prior art and overcomes the deficiencies of the prior art.

The present invention provides for a patient placement device for use in a diagnostic imaging and/or therapeutic, such as oncological, treatment setting. The invention is a device that may be easily adjusted to accommodate variations in head size and still allow for consistent and accurate patient placement or centering. The current invention may also facilitate the placement of subjects (both compliant and non-compliant) from preparation areas into the diagnostic imaging or oncology system thereby potentially saving time and resources. One particular, but not exclusive, area of diagnostic imaging for the present invention is in neurological applications, where placement of the head is critical. One embodiment of the present invention is particularly suited to the unique requirements of pediatric diagnostic medical imaging in all modalities, and serves several of the functions outlined in the prior art, with a focus on addressing the additional, unique and demanding needs of Pediatric MR Imaging described below.

Exemplary embodiments of the invention include a patient table compatible board or placement device including an integrated or interchangeable head and neck cradle that provides clearance for other medical equipment and is divided into flexible sections or flaps that adjust to accommodate a range of head diameters, an integrated user-adjustable head motion reduction system for centering a patients head in the iso-center of a desired location using the flexible sections, and geometry and locator tabs that couple the board to particular shapes and imaging or therapeutic systems for providing a consistent and repeatable position of the patient.

Further exemplary embodiments of the invention include a head motion reduction and control system including a spring-loaded member that displaces the flexible sections of the head cradle and can be hidden from view, an actuation system with tactile feedback and limited range to eliminate the risk of over tensioning that routes the motion for this member via a cable and housing, through an integrated slot that obscures its function from view, and a control system that can be coupled to the imaging or therapeutic system and that releases in the event of an emergency.

Further exemplary embodiments of the invention include a method of positioning a compliant or non-compliant patient on the board, including integrated straps on the board for minimizing motion of the body below the chest, a defined curvature across the width and length of the board, and a conformal inner geometry within the head and neck cradle for automatically centering a patient's body and occipital section of the head along the central plane of the board.

Further exemplary embodiments of the invention include aids for transferring a patient to an imaging or therapeutic system, including a conformal shaped head and neck cradle for supporting the neck and head during insertion or extraction, a lip or raised edge to allow for easy tilting and sliding of the board from a gurney or other system, a flat base for limiting the lift angle of the board and integrated, interchangeable, and curved handles to minimize stress on wrists when moving the board.

Further exemplary embodiments of the invention include a method for locating other imaging or therapeutic equipment, including locator strips and docking ports that are integrated into the subject positioning board.

In one embodiment, the present invention provides for subject placement device, characterized in that the subject placement device comprises: (a) a board for receiving a body of the subject, and (b) a head cradle extending from said board, said head cradle being enabled to receive a range of head diameters.

In one embodiment of the subject placement device of the present invention, the board includes integrated handles.

In another embodiment of the subject placement device of the present invention, the board includes one or more locator means for accommodating the subject placement device and the subject relative to a diagnostic or therapeutic system.

In another embodiment of the subject placement device of the present invention, the board includes two opposite ends, two lateral sides extending from the two ends, a bottom surface and a top surface, wherein said top surface includes a curvature extending from each lateral side to a central longitudinal axis that connects the two opposite ends of the top surface.

In another embodiment of the subject placement device of the present invention, the bottom surface of the board is configured to couple with a table of a diagnostic or therapeutic system.

In another embodiment of the subject placement device of the present invention, the bottom surface includes a recess capable of receiving a diagnostic or therapeutic equipment. In one aspect of the present invention, the diagnostic or therapeutic equipment is a spine MR coil.

In another embodiment of the subject placement device of the present invention, the bottom surface includes a flat surface adjacent to one of the opposite ends disposed at an angle relative to the mid portion of the bottom surface.

In another embodiment of the subject placement device of the present invention, the board includes one or more connectors for connecting the board to a diagnostic or therapeutic device. In one aspect of the invention, the diagnostic or therapeutic device is selected from the group consisting of a posterior coil for MRI and an anterior coil for MRI.

In another embodiment of the subject placement device of the present invention, the board includes one or more integrated slots for receiving strapping means for positioning and restraining the subject's body on the board.

In another embodiment of the subject placement device of the present invention, the head cradle is configured for coupling to a diagnostic or therapeutic system.

In another embodiment of the subject placement device of the present invention, the head cradle is integrated to the board.

In another embodiment of the subject placement device of the present invention, the head cradle is removably secured to the board.

In another embodiment of the subject placement device of the present invention, the head cradle includes a motion reduction system for reducing head motion within the head.

In one embodiment of the subject placement device of the present invention, the head cradle comprises of a shell having a front opening adapted to receive the head of the subject, the shell having an outer surface configured to couple with a diagnostic or therapeutic apparatus, an inner surface configured to center the subject's head within the head cradle, and radially disposed slots extending from the front opening into the shell, said slots defining movable flaps configured for enabling said head cradle to receive a range of head diameters. In one aspect of the present invention, the diagnostic or therapeutic apparatus is an MRI head coil having a bore, and said head cradle is configured to fit within the bore of the MRI head coil.

In another embodiment of the subject placement device of the present invention, the motion reduction system is coupled to the outer surface of each movable flap for controlling movement of the movable flap relative to the subject's head.

In another embodiment of the subject placement device of the present invention, the motion reduction system comprises of: (i) a spring-loaded member, (ii) a housing mounted to the movable flap, the housing for receiving the spring-loaded member, (iii) a cord having a first end operatively connected to an end portion of said spring-loaded member and a second end extending from the housing, and (iv) a substrate surface disposed relative to the housing whereby upon actuation of the second end of the cord the mid section of the spring-loaded member buckles away from the flap and comes into contact with the substrate surface thereby displacing the movable flap inwards. In one aspect of the present invention, the substrate surface comprises a panel attached to the outer surface of the head cradle. In another aspect of the present invention, the substrate surface is provided by a surface of the diagnostic or therapeutic system when the head cradle docks into the diagnostic or therapeutic apparatus.

In another embodiment of the subject placement device of the present invention, the motion reduction system further comprises a (v) an opening on the board for receiving the other end of the cord, and (vi) a network of slots integrated in the board for routing the cord from the housing to the opening.

In another embodiment of the subject placement device of the present invention, the second end of the cord is connected to a tab for the motion reduction control system, said tab extending from the opening and including a fastener for attachment to the diagnostic or therapeutic apparatus.

In another embodiment of the subject placement device of the present invention, the head cradle includes a neck cradle configured to conform to the subject's neck.

In another embodiment of the subject placement device of the present invention, the subject is a pediatric subject and wherein said board and said head cradle are sized and configured to receive the pediatric subject.

In another embodiment of the subject placement device of the present invention, the subject is a child from birth up to the age of around 6 years.

In another embodiment of the subject placement device of the present invention, the board can receive a subject weighing no more than 100 pounds.

In another embodiment of the subject placement device of the present invention, the board can receive a subject weighing no more than 45 pounds.

In another embodiment of the subject placement device of the present invention, the range of head diameters is between 9 and 16 centimeters.

In another embodiment of the subject placement device of the present invention, the range of head diameters is between 16 and 25 centimeters.

In another embodiment of the subject placement device of the present invention, the subject is a non-compliant subject.

In another embodiment of the subject placement device of the present invention, the subject placement device is made of a material that is compatible with a diagnostic or therapeutic system.

The above and other objects, features and advantages of the present invention will become apparent from the following description read in conjunction with the accompanying drawings, in which like reference numerals designate the same elements.

### BRIEF DESCRIPTION OF THE FIGURES

The following figures illustrate various aspects and embodiments of the invention. Referring now to the drawings wherein like elements are numbered alike in the several figures:

FIG. 1 illustrates a side perspective view of a portion of an exemplary embodiment of a patient positioning system including a patient positioning board, head and neck cradle, integrated restraint mechanisms and a patient in accordance to one embodiment of the present invention;

FIG. 2 illustrates a side perspective view of the portion of the system in FIG. 1 without the patient and placed on an imaging/therapeutic table with an imaging apparatus, in this case a conformal MR head coil;

FIG. 3 illustrates a side cross-sectional view of the positioning and imaging/therapeutic system in FIG. 2;

FIG. 4 illustrates a side perspective of the head and neck cradle of FIG. 1 with the patient and foam removed;

FIG. 5 illustrates a bottom plan view of the positioning system of FIG. 1;

FIG. 6 illustrates a front plan view of the head/neck cradle and motion reduction system of FIG. 1;

FIG. 7 illustrates another side perspective of the head/neck cradle and motion reduction system of FIG. 1;

FIG. 8 illustrates a side perspective of a cross-section of the control housing of FIG. 5;

FIG. 9 illustrates a side perspective view of a portion of an exemplary embodiment of the motion reduction system mechanism for use with the head cradle of FIG. 1;

FIG. 9a illustrates a front plan view of a portion of an exemplary embodiment of the motion reduction system mechanism and control for use with the head cradle of FIG. 1;

FIGS. 10a, 10b and 10c illustrate a side plan view of a portion of an exemplary embodiment of the spring of the motion reduction system of FIG. 9;

FIG. 11 illustrates another front plan view of a portion of an exemplary embodiment of the motion reduction system mechanism and control for use with the head cradle of FIG. 1;

FIG. 12 illustrates another side perspective view of a portion of an exemplary embodiment of the motion reduction system of FIG. 9;

FIG. 13 illustrates a side perspective view of a portion of an exemplary embodiment of the motion reduction system, head cradle and positioning of FIG. 1;

FIGS. 14a, 14b, 14c and 14d illustrate are side perspective view of a portion of exemplary embodiments of the spring-loaded members for the motion reduction system of the present invention;

FIG. 15 illustrates a side perspective view of an exemplary embodiment of the patient positioning system including a piece of additional imaging or therapeutic equipment;

FIG. 16 illustrates a side perspective view of a portion of an exemplary embodiment of the head cradle and motion reduction system in accordance to one embodiment of the present invention; and

FIG. 17 illustrates a side perspective view of the portion of the system in FIG. 1 without the patient and placed on an imaging/therapeutic table in an MR imaging system with an imaging apparatus, in this case a conformal MR head coil.

### DETAILED DESCRIPTION OF THE INVENTION

In this specification and in the claims that follow, reference will be made to a number of terms that shall be defined to have the meanings below. All numerical designations, e.g., dimensions and weight, including ranges, are approximations that typically may be varied (+) or (−) by increments of 0.1, 1.0, or 10.0, as appropriate. All numerical designations may be understood as preceded by the term “about”.

The singular form “a”, “an”, and “the” includes plural references unless the context clearly dictates otherwise.

The term “comprising” means any recited elements are necessarily included and other elements may optionally be included. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

“Subject” refers to mammals, particularly humans, but also to animals such as simians, cattle, horses, dogs, cats, and rodents. The term “subject” includes patients who may suffer from a disease and normal subjects.

For the purpose of this document, the terms “pediatric patient” or “pediatric subject” refers to an infant or child up to approximately 100 pounds. The definition includes a child from birth to up to the age of around 6 years. The definition includes also an infant or child of up to approximately 45 pounds.

The exemplary embodiments of systems, methods, and devices described herein enable fast and repeatable positioning of a subject, particularly a pediatric subject, in a diagnostic or therapeutic system. The exemplary embodiments of systems, methods and devices described herein enable also motion reduction of the subject's head and body. The diagnostic and therapeutic systems and methods described herein include multiple imaging and treatment modalities, including magnetic resonance imaging (MRI), computed tomography (CT), positron emission tomography (PET), single-photon emission computed tomography (SPECT), X-rays, radiation oncology, hybrid systems and so forth.

In one embodiment, the present invention provides for subject placement device, characterized in that the subject placement device comprises: (a) a board for receiving a body of the subject, and (b) a head cradle extending from said board, said head cradle being enabled to receive a range of head diameters.

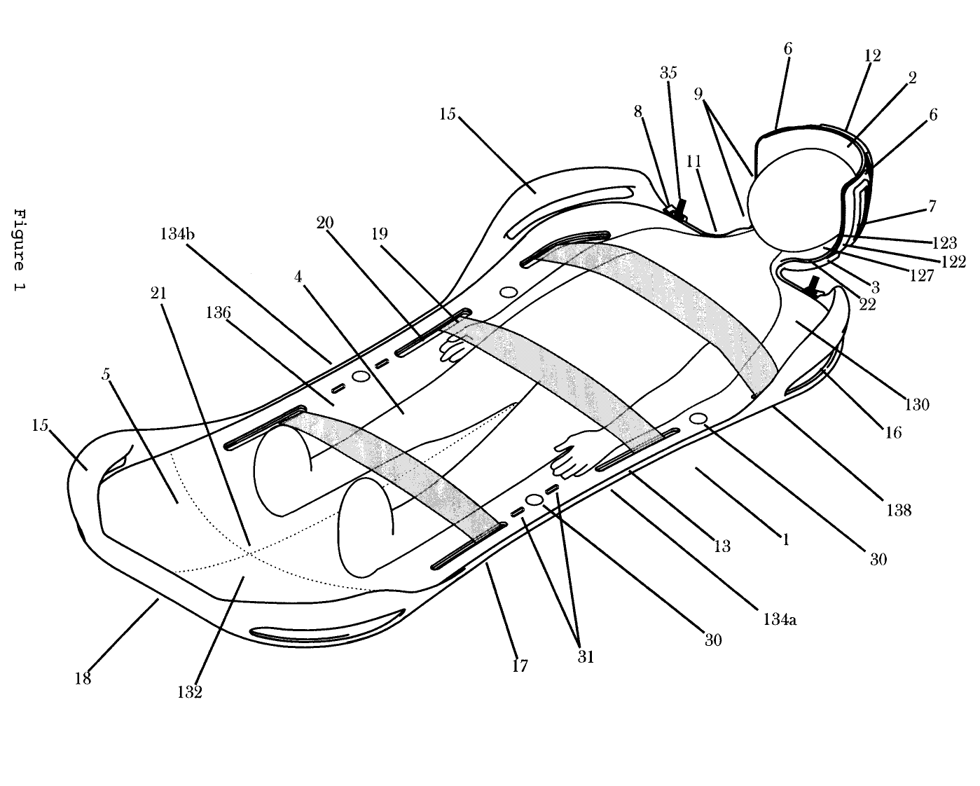

FIG. 1 shows an overall view of an exemplary embodiment of a subject's placement device 1 of the present invention. The subject's placement device 1 may include a positioning board 13 and a head cradle 2, which may include a neck cradle 3. FIG. 1 illustrates a subject 4 on the positioning board 13. The subject's head and neck may be positioned and supported by head and neck cradle 2, 3. In one embodiment of the present invention, the head and neck cradle 2, 3 may extend from and be integrated with the board section 13. In another embodiment, the head and neck cradle may be a separate, interchangeable structure having means for coupling with the board.

The positioning board 13 may include a first end 130, a second end 132, sides 134a,b extending in between the first 130 and second 132 ends, a top surface 136 and a bottom surface 138.

The head and neck cradle 2, 3 may extend from the first end 130 of the board 13. The head cradle 2 may be an open cradle and may include an outer geometry 12 and an inner geometry 22.

The subject may lie on the top surface 136 between the sides 134a,b, with the feet positioned closer to the second end 132, the subject's shoulders positioned adjacent to the first end 130 and the subject's head and neck positioned within the head and neck cradle 2, 3.

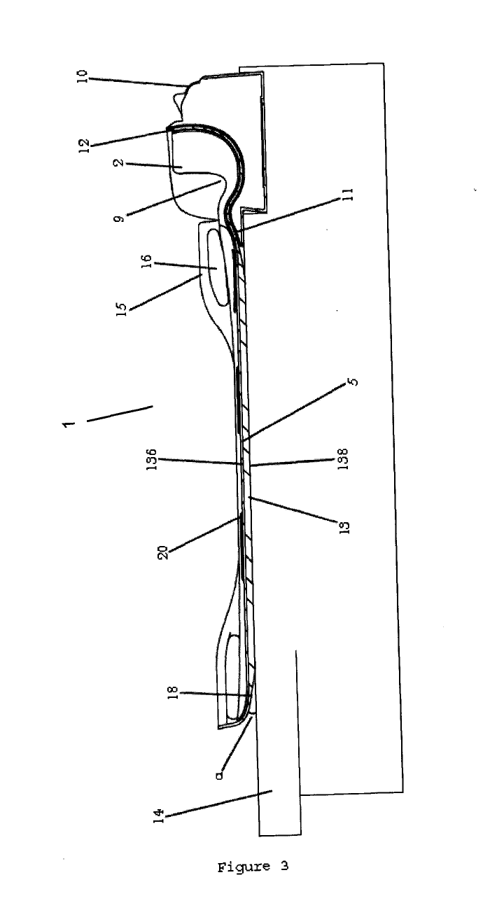

The subject's placement device 1 of the present invention may serve to facilitate the transfer of a subject by providing the board 13 with integrated curved handles 15 which may include openings 16 which may be tapered to help locate the hands of the handler, a raised lip 17 that allows the board to be tilted when sliding side to side. The bottom surface 138 may include a substantially flat surface 18 adjacent to the second end 132 that is set at a specified angle a (shown in FIG. 3) to the positioning board 13 that prevents the board from being lifted to an excessive angle. The board section 13 may include one or more integrated openings 20 to prevent the positioning straps 19 described bellow from interfering with sliding during patient transfer.

The positioning board 13 may include locator marks 30 that may be embossed or cut into the board. Other diagnostic or therapeutic equipment may be located relative to the subject 4 or positioning board 13 using the locator marks 30.

The subject's placement device 1 of the present invention may include features that aid in the positioning of a non-compliant patient, such as a child. The top surface 136 of the board 13 may include a curvature 21 from each side 134a,b towards the mid point of the board 13 that may be used to center the subject's body in the mid-plane of the board 13.

Slots 20 may be used for receiving straps 19, which may be integrated to the board 13. The subject's 4 body may be positioned and restrained using the integrated straps 19. For comfort, the positioning board 13 and head and neck cradle 2, 3 may be covered with a foam piece 5 that matches the shape of these components so as not to impede their function.

The head cradle 2 may be integrated to the board 13 or may be secured to the board 13 in such a way that it can be removed (i.e. removably secured), and is used to provide a subject with a comfortable and secure setting for diagnosis or therapy. The head cradle 2 is configured to receive or accommodate a range of head diameters; the head cradle 2 is enabled to automatically (i.e. without manual intervention) adjust to the head size of the subject (as opposed to enabling the subject or other person to adjust the cradle to the subject's head size) within a range of head sizes. The head cradle 2 may also be adjustable and include centering features so as to accommodate the subject's head in a desired, repeatable position within the cradle and to substantially restrain the subject's head within the head cradle so as to substantially prevent head's movement within the head cradle 2. Furthermore, the head cradle 2 may be configured to allow the technician of the diagnostic or therapeutic system maximum and convenient access to the subject/patient's neck and bead.

The bead cradle 2 may include a crown or shell 122 and a neck section 3 which attaches to first end 130 of the positioning board 13. The shell 122 may be comprised of an outer surface 12 and an inner surface 22. The geometry of the outer surface 12 of the shell 122 may be designed to couple with the diagnostic or therapeutic apparatus. For example, the outer surface 12 may be configured to dock within the bore of an MRI head coil, thereby avoiding the need of having MRI head coils of different sizes.

The shell 122 includes a peripheral border 123 which defines a front opening 127 for receiving the subject's head. The shell 122 may include radially disposed slots 27 extending from the front opening 127. The slots or cut-outs 27 define movable flaps 6 that enable the head cradle 2 to receive a range of head diameters.

The inner conformal geometry of the inner surface 22 of the head cradle 2 may be configured to locate the occipital part of the head in the mid-plane of the head cradle 2. The head cradle 2 may also include flaps or flexible sections 6 for enabling the head cradle to receive a range of head diameters. The flexible sections 6 may serve to provide support to the patient's head, and to provide a support substrate for a motion reduction system 7 for controlling movement of the flaps 6 as described herein bellow. Access for a technician's hands, A/V or other equipment may be provided around the subject's head using appropriate cutouts 9 in the head cradle 2.

In one embodiment not shown in the figures, the head cradle may include an attachment means adapted to releasably attach the head cradle to the positioning board.

The movement of the subject's head may be substantially restrained using the motion reduction system 7 which, in one aspect, may be integrated to the system 1 of the present invention. The motion reduction system 7 may work in conjunction with the movement of the flexible sections, or “flaps” 6 of the head cradle 2—the motion reduction system 7 may be actuated using control tabs 35 that are part of the motion reduction control system 8.

The subject's placement device 1, and thereby the subject 4, is located in an imaging or therapeutic system through the outer, conformal geometry of the head 12 and neck 11, and also locator tabs 31 that correspond to mounting points on the diagnostic or therapeutic system.

In one aspect of the present invention, the board and head cradle of the placement device of the present invention may be sized according to common anthropometric data.

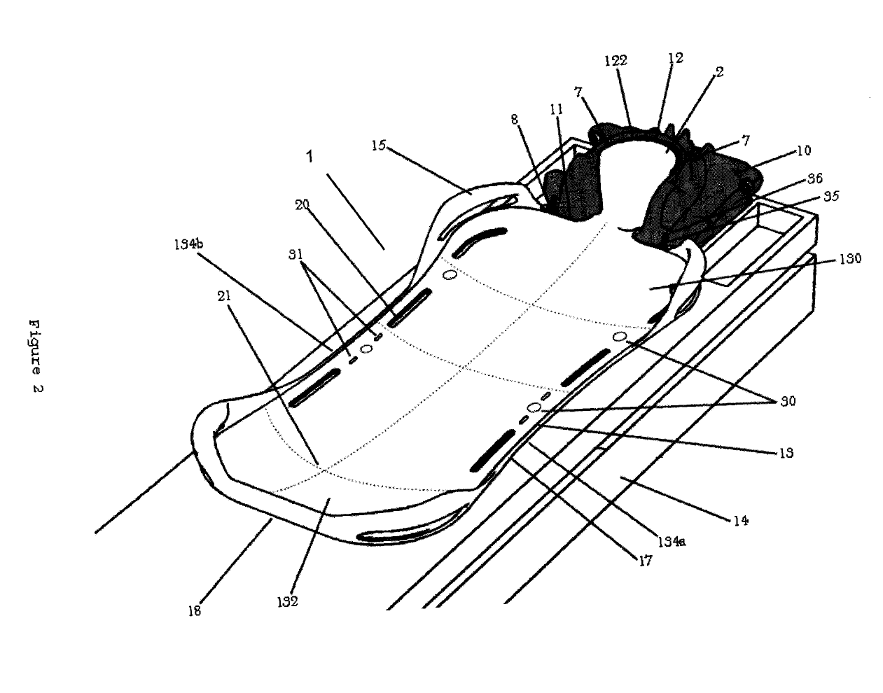

FIG. 2 illustrates the subject's placement device 1 as in FIG. 1 docked onto a patient table 14 of a diagnostic or therapeutic apparatus (or any form of reference geometry) 10, without the patient 4. The outer geometry 12, 11 of the head and neck cradle 2, 3 may be designed to be received by a bore within apparatus 10 so as to couple or dock with apparatus 10, which may be the head coil of a MRI machine, thereby locating the positioning board 13 and, in one embodiment, the apparatus 10 may serve to provide a surface for the motion reduction system 7 to work with.

After actuating the motion reduction system 7, the control tabs 35 for the motion reduction control system 8 may be attached to the diagnostic or therapeutic apparatus 10 using a fastener, including mechanical fasteners such as hooks and loops 36 or any other fastening means compatible with the apparatus 10. Normal removal may involve releasing control tab 35 from hook and loop 36 prior to extracting the subject's placement device. In cases of emergency or operator error, i.e. forgetting to release the control tab 35 from hook and loop 36, the control tabs 35 would automatically release from the imaging or therapeutic apparatus 10 through the act of lifting the subject's placement device 1 up and onto the patient table 14, eliminating the risk of equipment damage or patient injury.

FIG. 17 illustrates the subject's placement device 1 docked onto a patient table 14 as in FIG. 2, but shows it within an MR imaging system 55 and docked into an imaging apparatus, in this case a conformal MR head coil 53.

FIG. 3 shows a cross-section of the subject's placement device 1 of the present invention, a patient table 14 and a diagnostic or therapeutic apparatus 10 as in FIG. 2. This view in particular shows the outer geometry of the head and neck cradle 12, 11 matching that of the diagnostic or therapeutic apparatus (or any reference geometry) to locate the subject's placement device 1 in a repeatable manner. This view also shows the angle of the flat surface 18 that provides a reference when tilting the patient positioning board 13. Finally, this view shows tapered openings 16 of the curved handles 15 to help locate the hands.

FIG. 4 shows an overall view of the top half of the subject's placement device 1 as in FIG. 1, but with the fitted foam 5 removed. This view illustrates cut-outs 27 which may be designed to create the flexible sections or “flaps” 6 of the head cradle 2. The cut-outs 27 may be designed to create a constant spring rate for the flexible sections 6, and also to minimize stresses on the head cradle 2 when it is dropped. The flexible sections 6 have slots 25 that serve as (but are not limited to) mount points for the motion reduction system 7.

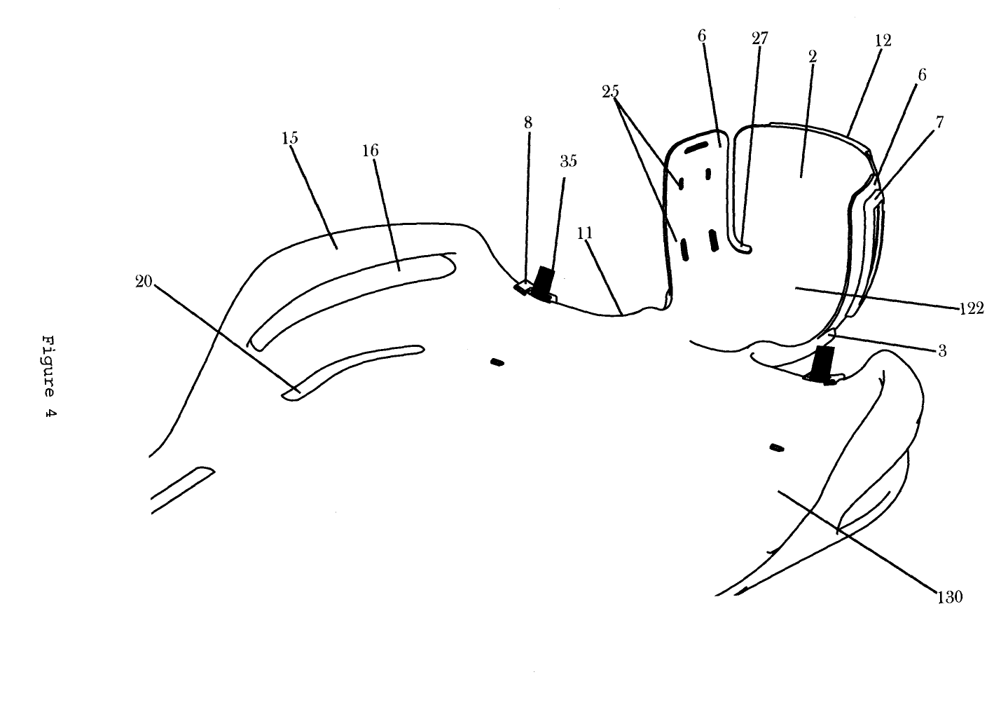

FIG. 5 shows a bottom plan view of the subject subject's placement device 1 as in FIG. 1. The bottom surface 138 of the subject positioning board 13 includes a flat surface adjacent to the second end 132 at an angle a that may be covered in a slippery material or coating 29 to aid in sliding of the board on to the diagnostic or therapeutic apparatus. The middle portion of the subject positioning board 13 may include a recess or well 38 that can accommodate an insert such as a blank or imaging equipment, such as a spine MR coil. The sides 134a,b of the positioning board 13 may include a curved profile or “hour-glass” shape 28 that allows the board to rest against the technician or operator during transfer operations. This view also further shows the conformal outer geometry 12 of the head cradle 2. FIG. 5 further illustrates the motion reduction control system 8, which may be comprised of a control means, such as cord or cable 42 having one end operatively connected to actuating tabs 35 and another end operatively linked the motion reduction system 7, whereby actuation of the tabs 35 (for example by pulling the tabs 35) results in actuation of the motion reduction system 7 as explained herein bellow. In one embodiment, as illustrated in FIG. 5, the board 13 may include an integrated slot network for cord or cable 42 routing. The integrated slot for cabling routing 33 may be used to route the control for the motion reduction system 7 to any location on the positioning board 13 using a hollowed out housing or tube 32 that can be covered for concealment/mounting. In the exemplary embodiment of FIG. 5, the motion reduction system controls 8 are located in the shoulder area or the handles 34,

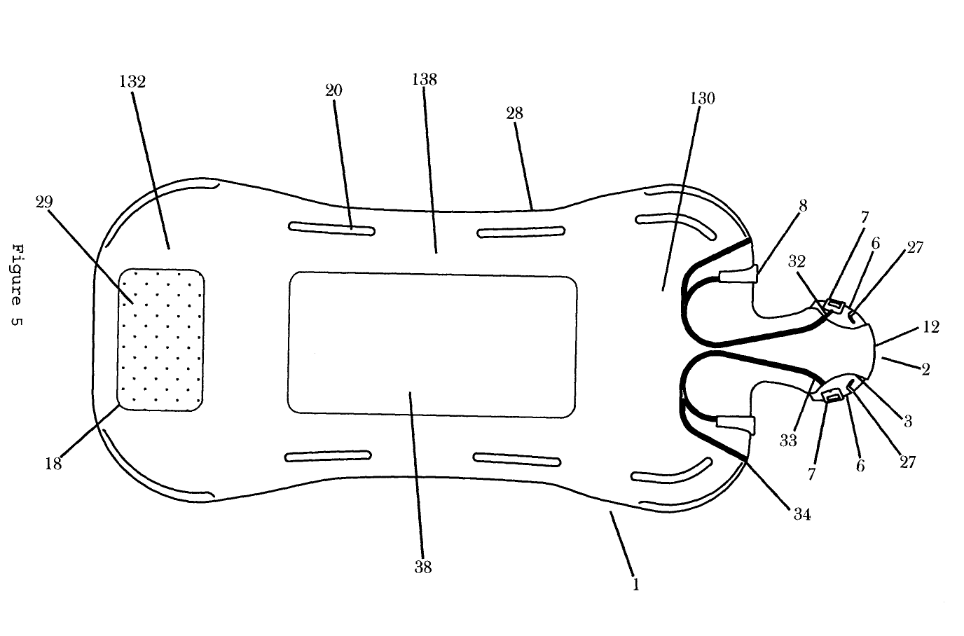

FIG. 6 shows a front plan view of the subject's placement device 1, as in FIG. 1.

The patient positioning board 13 has an angled “lip” 17 that allows the board to be tilted and slid during transfer operations. This view also further shows the conformal outer geometry 12 of the integrated (or interchangeable) head cradle 2. The control tabs 35 for the motion reduction system 7, as well as one embodiment for their fastening 36, (in this case hooks and loops) are on the left and right of the integrated head and neck cradle 2, 3. The integrated slot for routing 34 to the handles 15 is also shown.

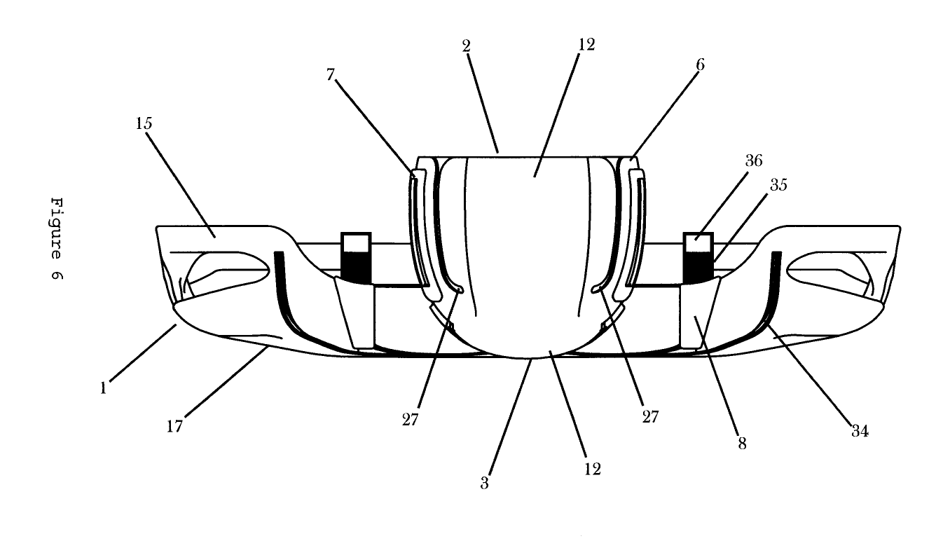

FIG. 7 shows a detailed view of the motion reduction system 7 and its integration with the subject's placement device 1 from FIG. 1. The motion reduction system 7 may include a housing 45 which may be mounted to the flexible sections 6 of the head cradle 2, and a spring-loaded member 40 which may be actuated using a control means such as a cord or cable 42 or a strap that is mounted to the mount point for the control cord 43, then routed through a slot 41 in the spring-loaded member 40, and through a housing or tube 32 that may be seated in an integrated slot 33 that is directed to the shoulder area of the first end 130 where a control tab 35 is connected to the other end of the control cord 42. It should be understood that the integrated slot may be directed to any other convenient are of the board.

Access for a technician's hands, A/V or other equipment may be provided around the face using appropriate cutouts 9 in the head and neck cradle 2, 3.

FIG. 8 shows a cross section of the housing or tube 32 of FIGS. 7 and 5 and the control cord 42 that passes through this housing.

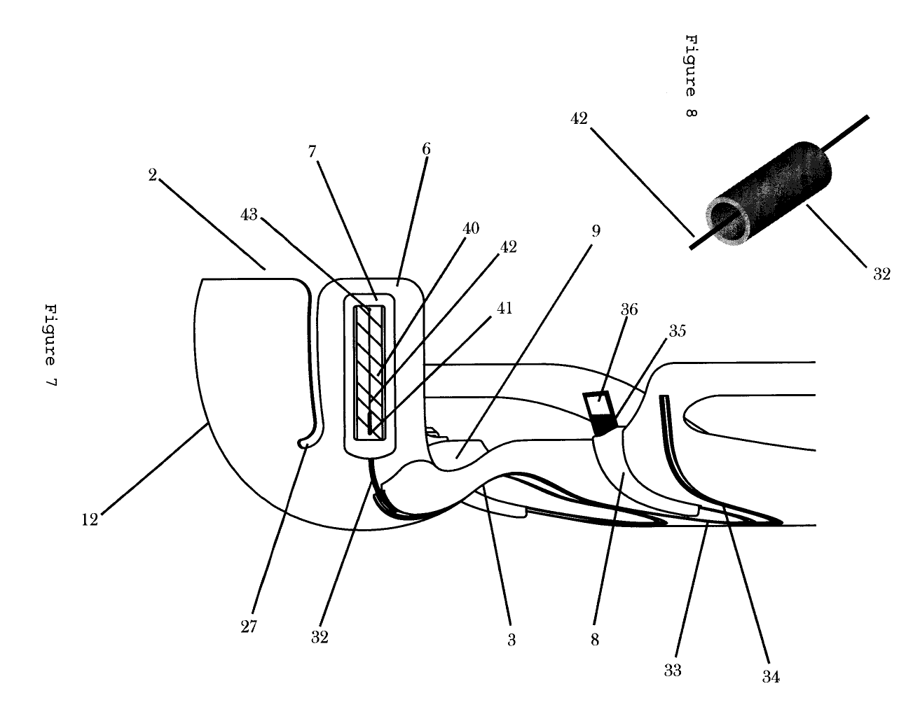

FIGS. 9 and 9a show a detailed view of the components of the motion reduction system 7 of FIG. 1. The system comprises a T-shaped spring-loaded member or “t-spring” 40, a housing 45 for receiving the t-spring 40, and controlling means such as cord 42.

As illustrated in FIG. 9, the T-shaped spring-loaded member comprises of a substantially flat main body 400 having sides 402 and two tabs 44 extending from one end 404 of the main body 400 so as to form the “T” shaped member. The main body 400 may include a slot 41 that allows clearance for the control cord 42 and a mount point 43, which may take the form of a hole.

The two tabs 44 may allow the spring-loaded member to be held in place by the housing 45 by two cut-outs 46. The t-spring 40 also has a mount point 43 that is a hole for looping the control cord 42 to the spring, thereby coupling the two components.

The housing 45 for the motion control mechanism comprises of a first end 900 and a second end 902 and sides 904. The housing 45 may include integrated snaps 49 on the second end 902 that couple with cut-outs 25 on the head cradle 2 to mount the system 7, an opening 48 for receiving the spring-loaded member 40, cut-outs 46 on the first end 900 for holding the t-spring 40 in place, slots 51 along the sides 904 to guide spring motion and an opening 47 on the first end 900 to allow access for the control cord 42. The inner surface of the housing 45 has a matching geometry 50 that tightly mates with the outer geometry of the head cradle 12.

FIG. 9a shows a front plan view of the assembled motion reduction system 7, showing its connection via control cord 42 to the control tabs 35 with its mounting system 36 (in this embodiment, hooks and loops).

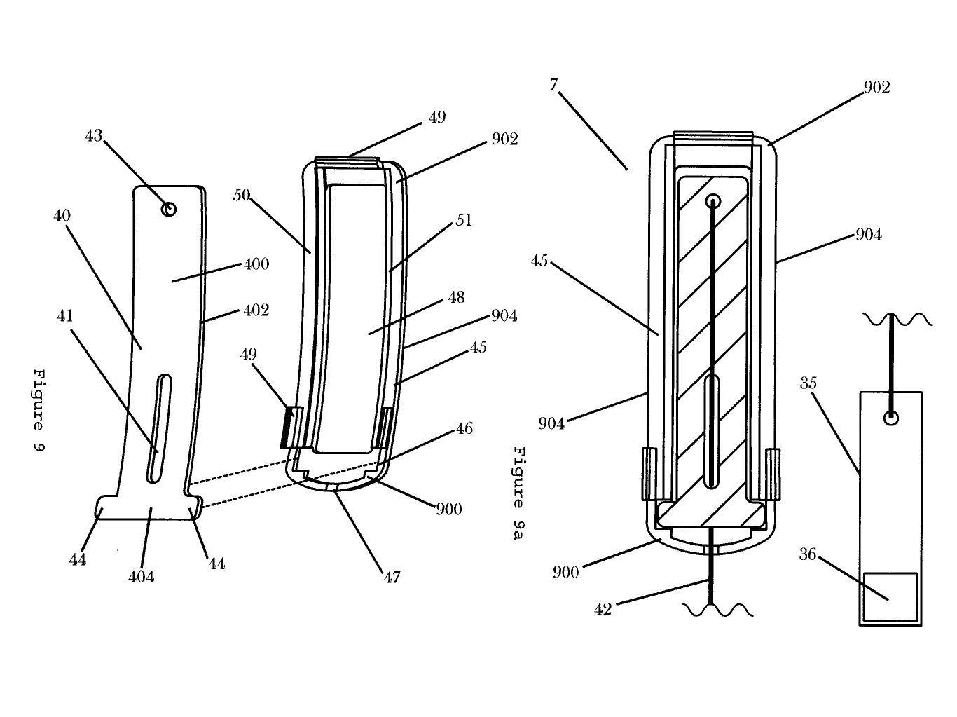

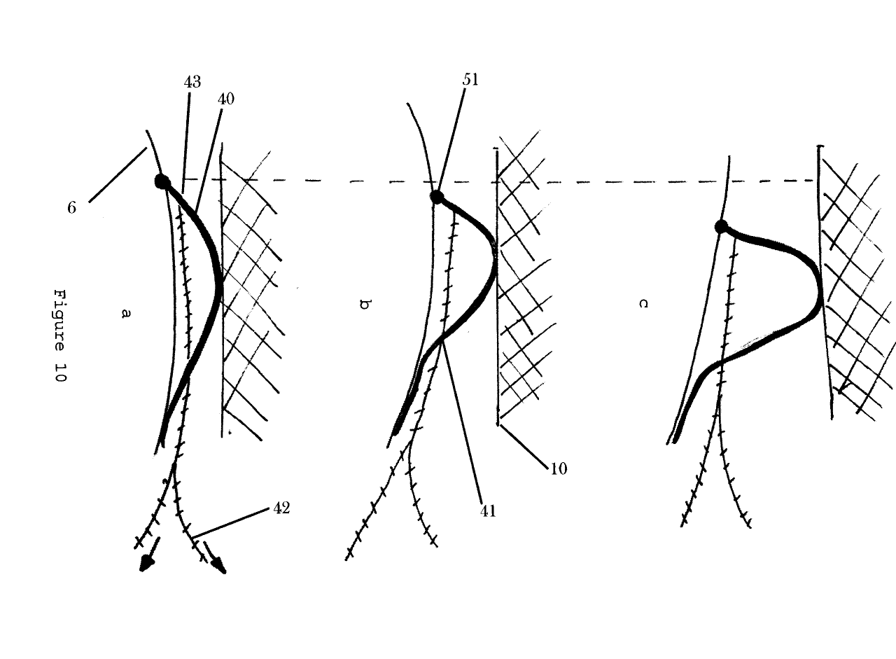

FIGS. 10a,b,c shows three side plan views that show schematically how the “t-spring” 40 of the motion reduction system 7 may be used in conjunction with the adjustable flexible sections 6 of the head cradle 2. The control cord 42 may be actuated or pulled, which, through its coupling to the t-spring 40 at the mount hole 43, causes the spring-loaded member 40 to buckle outwards (FIG. 10a). The control cord 42 moves through the opening 41 in the spring 40 (FIG. 10b). As the spring-loaded member 40 buckles, it travels through the guiding slot 51 in the housing 45 and pushes against a surface of the imaging apparatus or therapeutic apparatus 10 or the surface of a reference geometry, thereby moving the flexible section 6 inwards against the patient's 4 head (FIG. 10c).

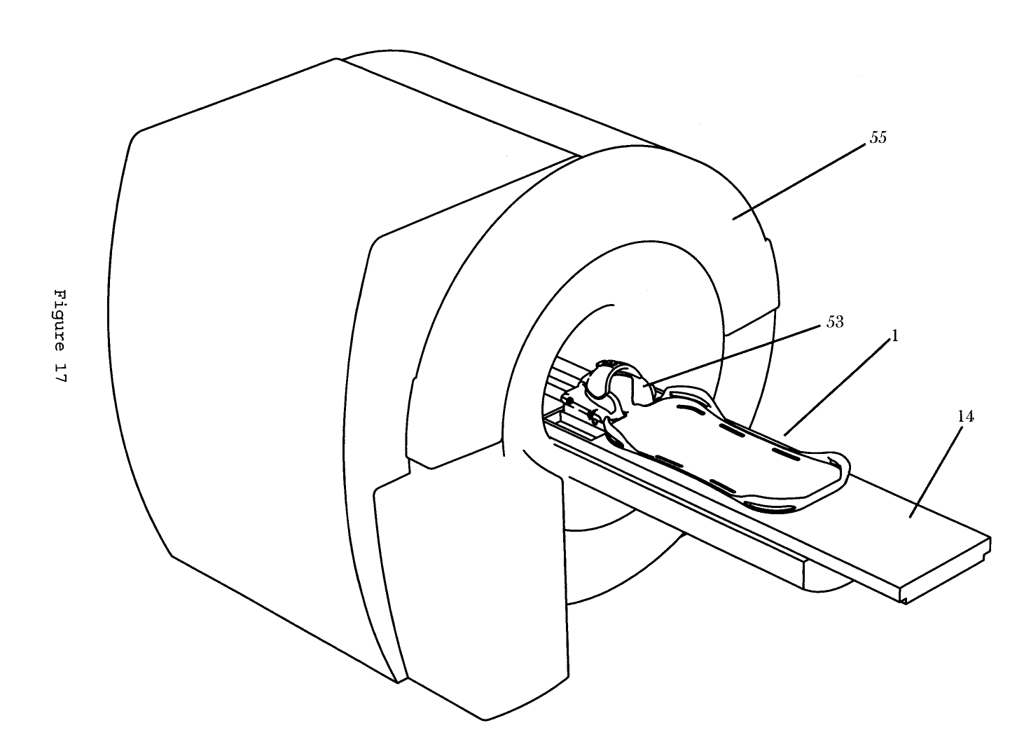

FIG. 16 illustrates another embodiment for a self containing motion reduction system that does not depend on the surface of imaging or therapeutic apparatus. In this embodiment, a panel 160 is coupled to the outer surface 164 of the head cradle 162. The panel 162 provides the substrate surface for the spring-loaded member (not shown as is it covered by the panel 162) to push against, thereby moving the flexible section inwards against the patient's head.

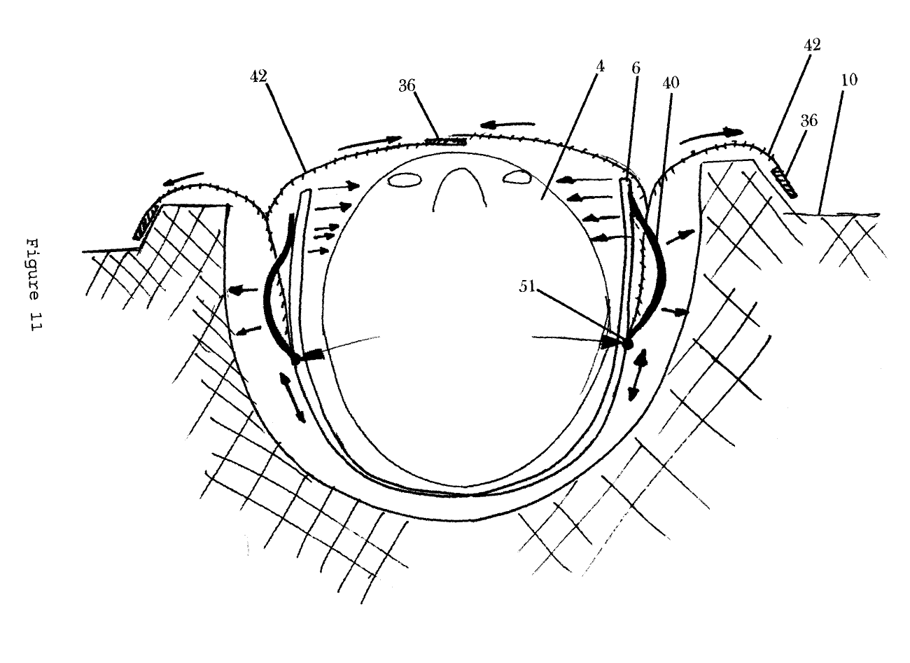

FIG. 11 shows a schematic front plan view that elaborates on the operation of the motion reduction system 7 in FIG. 10. Here, after actuating the t-spring 40 to move the flexible sections 6 against the patient's head 4, the control cord 42 is coupled to the imaging apparatus or reference geometry using the mounting system (hooks and loops in the preferred embodiment) 36. Upon release of the control cord's 42 mounting system 36, the spring rate of the flexible sections 6 and the spring rate of the “t-spring” 40 cause the motion reduction system 7 to return to its neutral state—this feature may be important in the release of the motion reduction system 7 in the case of an emergency.

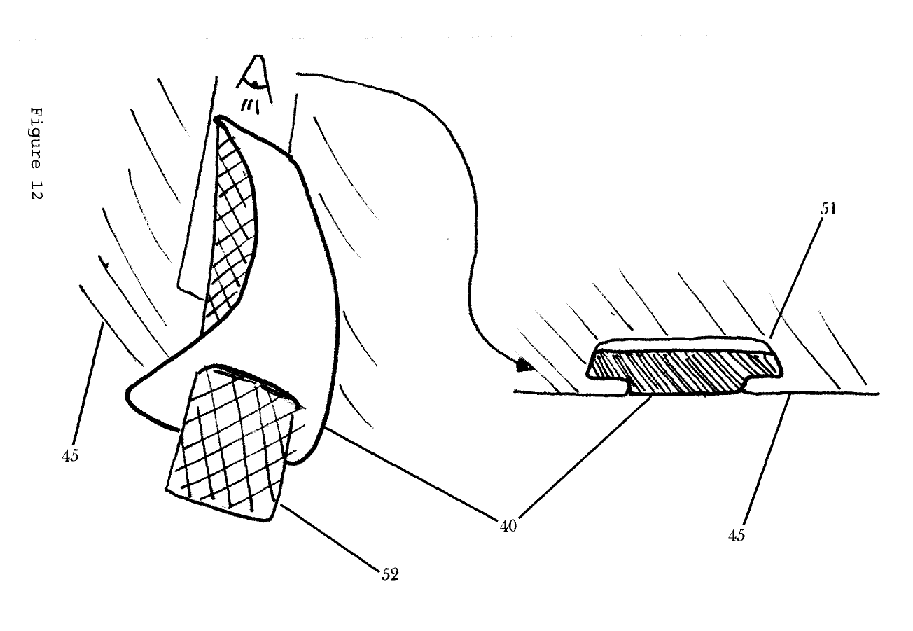

FIG. 12 shows an overall view of an embodiment of the t-spring 40 in the motion reduction system 7 of FIG. 1, where the spring-loaded member 40 is shaped such that it rides in the integrated slot 51 of the housing 45 for the motional reduction mechanism. In this embodiment, the spring-loaded member 40 is actuated using a strap 52 instead of a cord 42.

FIGS. 13, 14 and 15 illustrate other exemplary embodiments of the patient subject's placement device of the present invention. For convenience, the reference numbers used in FIGS. 1-12 will be used in FIGS. 13-15 for like parts.

FIG. 13 shows another exemplary embodiment of the subject's placement device 1 of the present invention. The patient subject's placement device 1 includes a sliding chin pad and positioner 60 and a motion reduction system 7 that is actuated using a strap 52 that goes across the head cradle 2. In this embodiment, the cut-out 27 to create the flexible sections 6 is vertical.

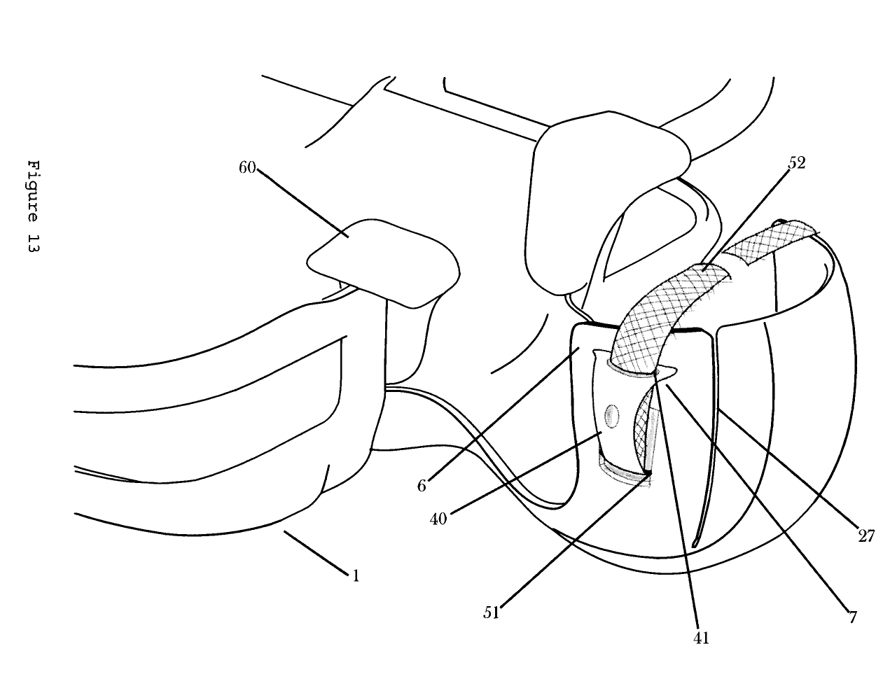

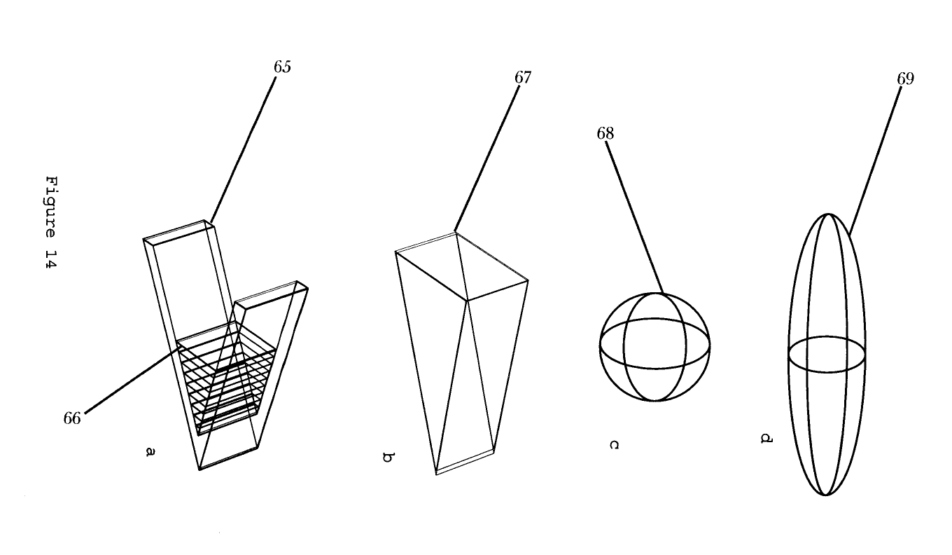

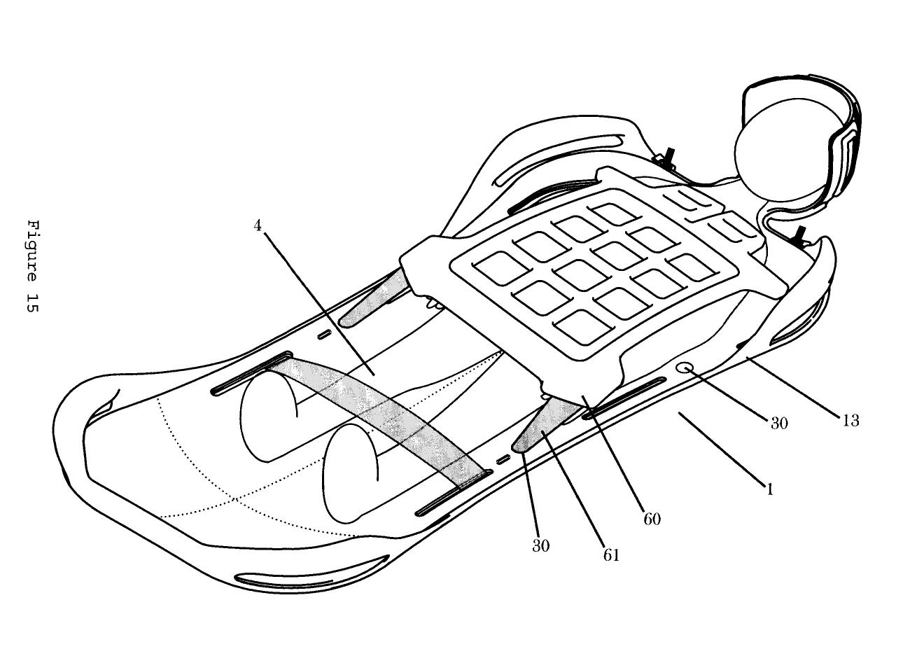

FIG. 14 illustrates a number of variations for items that could take the place of the “t-spring” 40 shown in FIGS. 7 and 9. FIG. 14a illustrates a v-shaped hard piece 65 that has an inner spring or other compliant material 66 that compresses and provides spring-back force against the reference geometry 10. FIG. 14b illustrates a solid v-shaped wedge that would be wedged between the flexible sections 6 and the reference geometry 10. FIGS. 14c and 14d illustrate spherical and ovoid shapes 68, 69 that can be made of solid or compliant material, and would be wedged between the flexible sections 6 and the reference geometry 10.

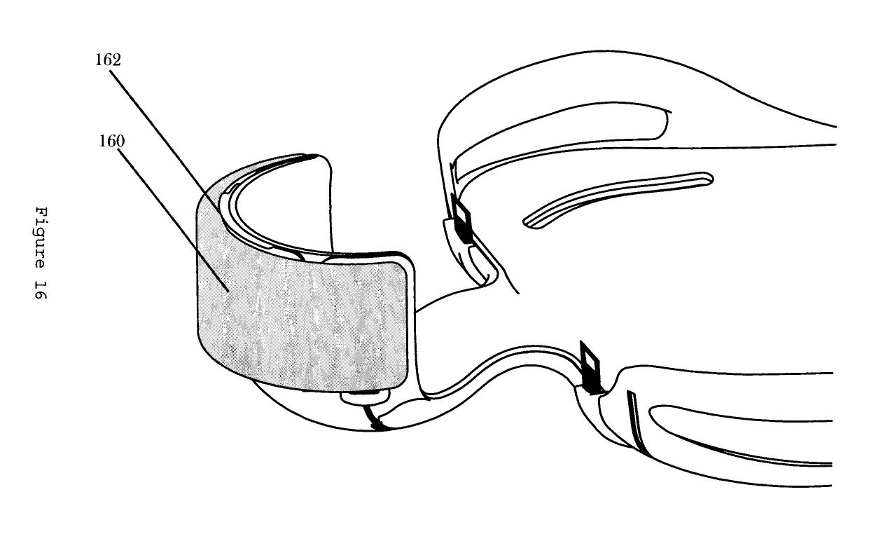

FIG. 15 shows another embodiment of the invention, where the subject's placement device 1 may be used in conjunction with another imaging or therapeutic device 60 (in this embodiment a posterior coil/anterior coil for MRI) which may be attached to the integrated locator or connector points 30 on the positioner board 13 via mounting hardware 61 that can be fabric or solid material. This embodiment allows for maximum user/application flexibility to accommodate a particular procedure.

For X-ray, MRI or CT compatibility, the subject's placement device of the present invention may be made of X-ray, MR/CT compatible materials. The subject's placement device may be manufactured, for example, using thermo-forming or any additive manufacturing process, or injection moulding, casting and so forth. In particular, additive manufacturing can allow for custom, conformal shapes that aid in docking of the subject's placement device to the diagnostic/therapeutic device.

One embodiment of the present invention is particularly suited to the unique requirements of pediatric diagnostic medical imaging and serves several of the functions outlined in the prior art, with a focus on addressing the additional, unique and demanding needs in pediatric diagnostic imaging, in particular the large variations in anthropometric sizing and non-compliant patients. The time required to accurately place a child's head in a conformal RF coil can be greatly reduced through the use of the proposed invention. The flexible head cradle section of the present invention being an open design, provides access to significant areas of the child's head, thereby permitting the placement of the attendant MRI technician's hands on the head for ease of child placement in the cradle. The cradle is designed to work with the conformal RF coil for consistent, optimal patient bead placement at the iso-center of the coil. This is achieved through the use of the flexible, open head cradle section to accommodate a range of head diameters, and an integrated user- adjustable head motion reduction system for automatically centering a child's head, such as the center of a RF bead coil in MRI or a head cradle for therapeutic treatments.

Advantages of the present invention include: a design of a patient preparation, placement, and positioning device which increases accessibility for subjects, decreases head motion and motion-related imaging artifacts, facilitates the preparation and placement of subjects (compliant and non-compliant) to and from a gurney and imaging system patient table (and in MR, the RF coil), allows patient preparation outside of the exam room if desired, and provides ease of setup for the imaging technologist. In addition, the present invention has advantages as a pediatric device because it: (1) is sized appropriately for small children, and the related Diagnostic Imaging and Oncology equipment; (2) can handle the size variations of small children; (3) is designed to position non-compliant patients; (4) hides the functional elements to increase child acceptance; (5) allows for open access to the patient for intubation tubes, monitoring, audio/visual equipment for fMRI (functional MRI) or monitoring, and (6) allows for rapid and easy extraction in emergency situations.

While certain embodiments have been illustrated and described in the foregoing examples, it will be understood that changes and modifications can be made in the foregoing methods to practice the present technology in accordance with ordinary skill in the art without departing from the present technology in its broader aspects as defined in the following claims.

The inventions have been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

What is claimed is:

1. A subject placement device, characterized in that the subject placement device comprises: (a) a board for receiving a body of the subject, and (b) a head cradle extending from said board, said head cradle being enabled to receive a range of head diameters.

2. The subject placement device of claim 1, characterized in that the board includes integrated handles.

3. The subject placement device of claim 1, characterized in that the board includes one or more locator means for accommodating the subject placement device and the subject relative to a diagnostic or therapeutic system.

4. The subject placement device of claim 1, characterized in that the board includes two opposite ends, a mid portion between said two opposite ends, two lateral sides extending from the first and second ends, a bottom surface and a top surface, wherein said top surface includes a curvature extending from each lateral side to a central longitudinal axis that connects the two opposite ends of the top surface.

5. The subject placement device of claim 4, characterized in that the bottom surface is configured to couple with a table of a diagnostic or therapeutic system.

6. The subject placement device of claim 4, characterized in that the bottom surface includes a recess capable of receiving a diagnostic or therapeutic equipment.

7. The subject placement device of claim 6, characterized in that the diagnostic or therapeutic equipment is a spine MR coil.

8. The subject placement device of claim 4, characterized in that the bottom surface includes a flat surface adjacent to one of the opposite ends disposed at an angle relative to the mid portion of the bottom surface.

9. The subject placement device of claim 1, characterized in that the board includes one or more connectors for connecting the board to a diagnostic or therapeutic device.

10. The subject placement device of claim 9, characterized in that the diagnostic or therapeutic device is selected from the group consisting of a posterior coil for MRI and an anterior coil for MRI.

11. The subject placement device of claim 1, characterized in that the board includes one or more integrated slots for receiving strapping means for positioning and restraining the subject's body on the board.

12. The subject placement device of claim 1, characterized in that the head cradle is configured for coupling to a diagnostic or therapeutic system.

13. The subject placement device of claim 1, characterized in that the head cradle is integrated to the board.

14. The subject placement device of claim 1, characterized in that the head cradle is removably secured to the board.

15. The subject placement device of claim 1, characterized in that the head cradle includes a motion reduction system for reducing head motion within the head.

16. The subject placement device of claim 15, characterized in that the head cradle comprises of a shell having a front opening adapted to receive the head of the subject, the shell having an outer surface configured to couple with a diagnostic or therapeutic apparatus, an inner surface configured to center the subject's head within the head cradle, and radially disposed slots extending from the front opening into the shell, said slots defining movable flaps configured for enabling said bead cradle to receive a range of head diameters.

17. The subject placement device of claim 16, characterized in that the diagnostic or therapeutic apparatus is an MRI head coil having a bore, and wherein said head cradle is configured to fit within the bore of the MRI head coil.

18. The subject placement device of claim 16, characterized in that the motion reduction system is coupled to the outer surface of each movable flap for controlling movement of the movable flap relative to the subject's head.

19. The subject placement device of claim 18, characterized in that the motion reduction system comprises of: (i) a spring-loaded member, (ii) a housing mounted to the movable flap, the housing for receiving the spring-loaded member, (iii) a cord having a first end operatively connected to an end portion of said spring-loaded member and a second end extending from the housing, and (iv) a substrate surface disposed relative to the housing whereby upon actuation of the second end of the cord the mid section of the spring-loaded member buckles away from the flap and comes into contact with the substrate surface thereby displacing the movable flap inwards.

20. The subject placement device of claim 19, characterized in that the substrate surface comprises a panel attached to the outer surface of the head cradle.

21. The subject placement device of claim 19, characterized in that the substrate surface is provided by a surface of the diagnostic or therapeutic apparatus when the head cradle docks into the diagnostic or therapeutic apparatus.

22. The subject placement device of claim 19, characterized in that the motion reduction system further comprises a (v) an opening on the board for receiving the other end of the cord, and (vi) a network of slots integrated in the board for routing the cord from the housing to the opening.

23. The subject placement device of claim 22, characterized in that the second end is connected to a tab for the motion reduction control system, said tab extending from the opening and including a fastener for attachment to the diagnostic or therapeutic apparatus.

24. The subject placement device of claim 1, characterized in that said head cradle includes a neck cradle configured to conform to the subject's neck.

25. The subject placement device of any one of claims 1 to 24 wherein said subject placement device is a pediatric device, wherein said subject is a pediatric subject and wherein said board and said head cradle are sized and configured to receive the pediatric subject.

26. The subject placement device of claim 25, characterized in that said subject is a child from birth up to the age of around 6 years.

27. The subject placement device of any one of claims 1 to 24, characterized in that said board can receive a subject weighing no more than 100 pounds.

28. The subject placement device of any one of claims 1 to 24, characterized in that said board can receive a subject weighing no more than 45 pounds.

29. The subject placement device of any one of claims 1 to 24, characterized in that said range of head diameters is between 9 and 16 centimeters.

30. The subject placement device of any one of claims 1 to 24, characterized in that said range of head diameters is between 16 and 25 centimeters.

31. The subject placement device of any one of claims 1 to 24 wherein said subject is a non-compliant subject.

32. The subject placement device of any one of claims 1 to 31, characterized in that said subject placement device is made of a material that is compatible with a diagnostic or therapeutic system.
